(19) United States
(12) Patent Application Publication
(10) Pub. No.: US 2020/0129372 A1
(43) Pub. Date: Apr. 30, 2020

(54) BEAUTY DEVICE

(71) Applicant: LIFETRONS SWITZERLAND GROUP LIMITED, Tuen Mun (HK)

(72) Inventor: Chih-Ming TSENG, Tuen Mun (HK)

(21) Appl. No.: 16/659,903

(22) Filed: Oct. 22, 2019

(30) Foreign Application Priority Data
Oct. 31, 2018 (TW) 107214797

(51) Int. Cl. A61H 23/02; H02J 7/00; A61N 5/06

(52) U.S. Cl. CPC A61H 23/02; H02J 7/0042; A61N 5/0616; A61H 2201/10; A61N 2005/0626; A61H 2205/024; A61H 2201/5097; A61N 2005/0663; A61N 2005/0644; A61H 2205/021

(57) ABSTRACT

A beauty device includes a power bank and at least one beauty instrument. The power bank includes: a connecting interface which is configured on one side of the power bank, a touch switch which is configured next to the side of the connecting interface, a control module which connects with the connecting interface and the touch switch and a power output module which connects with the control module. A power output module is composed by connecting a power management module and a battery module and also the power management offers different power supply specifications. The beauty instrument has a beauty function output module and the beauty instrument connects with the head and the connecting portion. The power management module offers appropriate power supply specification to the beauty instrument so the power bank can be transformed from single function to the power bank with multiple functions.

### FIELD OF THE INVENTION

The present invention relates to the technical field of beauty instruments, and more particularly to a beauty device has multiple power supply source that can provide to the beauty instruments.

### BACKGROUND OF THE INVENTION

When the users perform the process of beauty care, they may need to use a variety of beauty instruments with different functions, for example: the use of negative ion function of the beauty instrument is to promote absorption of more collagen and elastin in order to initiate the cell to restore the skin elasticity so that wrinkles, edema and skin aging can be reduced and firmness of the skin can be improved. However, it is difficult to achieve the functions of color light treatment and massage at the same time. Therefore, even if the negative ion function of the beauty instrument can be applied when performing the process of beauty care, dark circles under the eyes and skin problems cannot be alleviate effectively. Thus, the beauty instruments with different functions need to be used to meet the requirement for both performing skin care and removing dark circles under the eyes.

However, with current technology on beauty instruments, only single power source can be used to provide a beauty instrument with solely massage function. Therefore, when the users need to use different beauty instruments for different functions, different power supply devices are required. Meanwhile, current power banks can only offer a single specification for the power supply. Therefore, it is not possible to interchange different beauty instruments with power banks with single power supply specification. It can be both costly and inconvenient to not solve the technical problem of offering power banks with multiple power supply specifications to supply multiple beauty devices.

### SUMMARY OF THE INVENTION

In order to overcome the inconvenience of using different power supply devices, the main purpose of the present invention is to provide a beauty device that offers multiple power supply specifications so that different beauty instruments can be interchanged freely with one single power bank and the users can apply this beauty device for facial care and getting rid of dark circles under the eyes.

According to the purpose mentioned above, the present invention provides a beauty device which includes a power bank and at least one beauty instrument. The power bank includes a connecting interface which is configured on one side of the power bank, a touch switch which is configured next to the side of the connecting interface, a control module which is configured inside of the power bank and connects with the connecting interface and the touch switch, and an power output module is composed of a power management module and a battery module which is configured inside of the power bank and connects with the control to offer different power supply specifications. A beauty instrument includes a head and a connecting portion opposite to the head. The beauty instrument has a beauty function output module and is connected to the head and the connecting portion, on the head of the beauty instrument, a metal layer is configured and is connected with the connecting portion. Accordingly, after the connecting portion of the beauty instrument is connected the interface of the power bank through the connecting portion, the power management module offers appropriate power supply according to specification of the beauty instrument.

For the preferred embodiment of the present invention, the connecting interface of the power bank and the connecting portion b of the beauty instrument are a USB interface.

For the preferred embodiment of the present invention, the beauty instrument further includes an identification module and also when the identification module is a Bluetooth module, the beauty instrument can match the different supply specifications of the power management module through the Bluetooth module.

For the preferred embodiment of the present invention, the touch switch on the power bank and the metal layer on the head of the beauty instrument can form a circuit through the human body to form another kind of switch for controlling the battery module of the power output module to provide the corresponding power supply to achieve the effect of energy savings.

According to the above purpose, the present invention provides another beauty device which includes a power bank and at least one beauty instrument. The power bank includes a connecting interface which is configured on one side of the power bank, a control module which is configured inside the power bank and connects with the connecting interface, and a power output module which is configured inside of the power bank and connects with the control module, in which the power output module is composed by a power management module and a battery module. A beauty instrument includes the head and the connecting portion opposite to the head, and further has a beauty function output module which respectively connects to the head and the connecting portion. The connecting portion of the beauty instrument connects the connecting interface of the power bank to activate the power management module of the power output module to provide appropriate power supply specifications to the beauty function output module of the beauty instrument.

For the preferred embodiment of the present invention, the connecting portion between the connecting interface of the power bank and the beauty instrument is a USB connector. For the preferred embodiment of the present invention, the touch switch on the power bank and the metal layer on the head of the beauty instrument can form a circuit through the human body to form another kind of switch for controlling the battery module in the power output module to provide the corresponding power to achieve the effect of energy savings.

In addition, comparing the beauty device mentioned above with current technology, the beneficial effects are: the power bank provides the power to the massage head with the negative ion, micro-vibration to perform the facial treatments, dark circles under the eyes treatments, and head scalps treatments, upgrading to the multiple beauty functions from a single beauty function. Negative ions promotes the absorption of skin care products, micro-vibration massages skin, and red light whitens the skin, therefore, the dark circles under the eyes and wrinkles can effectively alleviate to improve the hair quality. With the power bank function, the power bank can provide the power to the beauty device freely, and with multiple combinations of functions, such that the beauty instrument of the invention can greatly meet the needs of the consumers.

### BRIEF DESCRIPTION OF THE DRAWINGS

FIG. 1 is a schematic view showing the appearance of a specific embodiment of a power bank for the present invention.

FIG. 2 is a schematic view of a specific embodiment of a beauty device for the present invention.

FIG. 3 is a block diagram showing a specific embodiment of the beauty device of the present invention.

FIG. 4 is a block diagram showing another embodiment of the beauty device of the present invention.

### DETAILED DESCRIPTION

The advantages and features of the present invention can be more easily understood if referring to the exemplary embodiments and the accompanying drawings of the present invention. However, the intention is not to limit the present invention only to these embodiments set forth herein On the contrary, these provided embodiments are to offer a thorough and complete disclosure of the scope of the present invention.

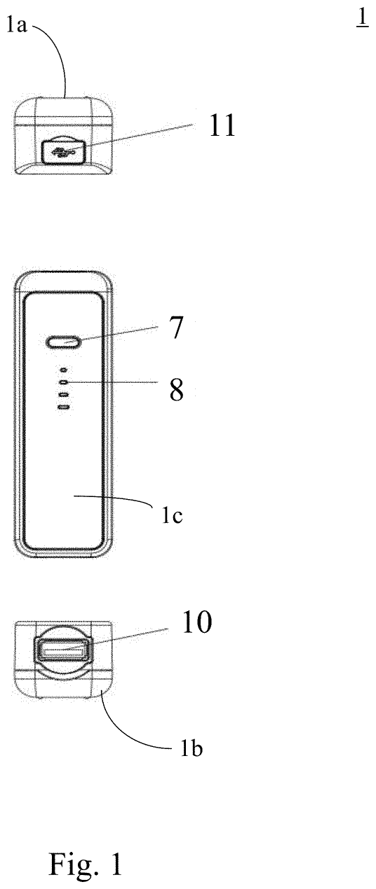

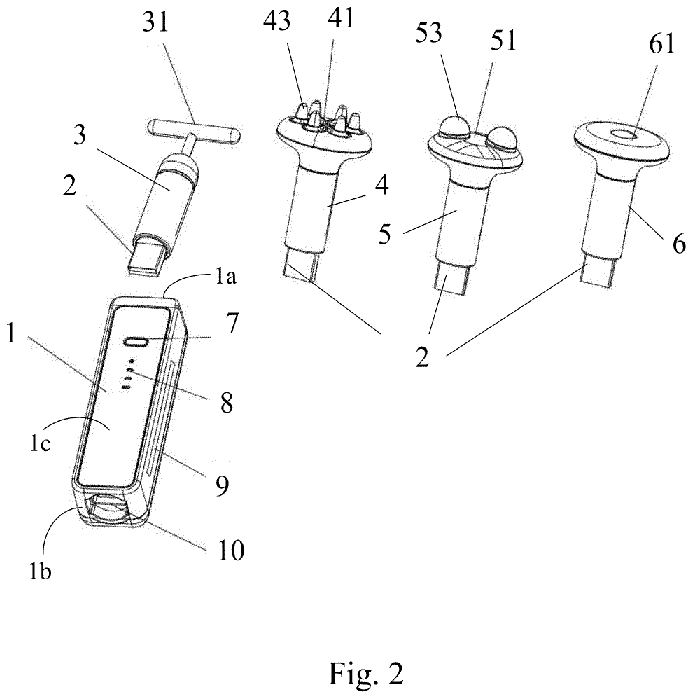

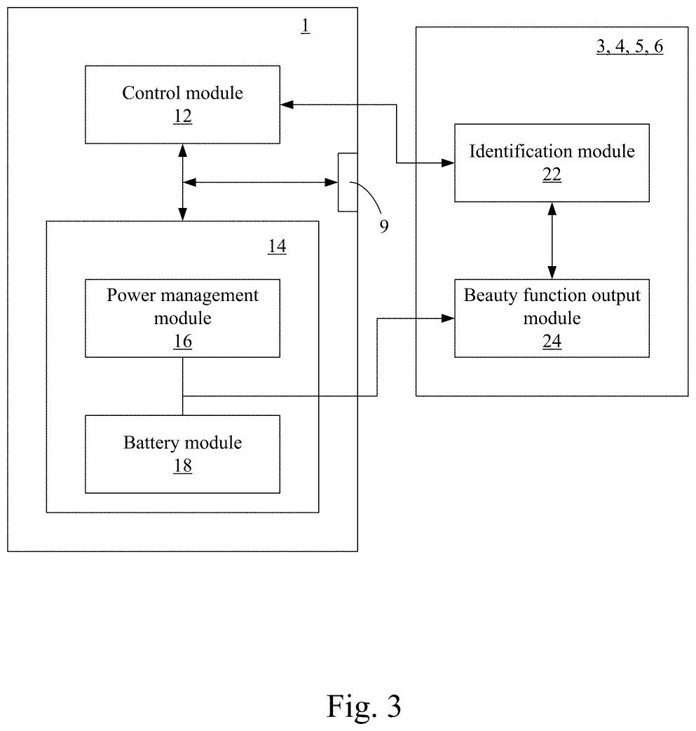

First, please refer to FIG. 1, which is a schematic view showing the appearance of a power bank of a specific embodiment for the present invention. As shown in FIG. 1, the power bank 1 in the present invention includes a connecting interface 10 at the top end 1b of the power bank 1, for example: an interface that can be connected to various Universal Serial Bus (USB) and can connect with the control module 12 (shown in FIG. 3). The bottom 1a of the power bank 1 has a recharging interfacell, when the power of the power bank 1 is insufficient, the battery of the power bank 1 can be recharged through the recharging interface 11, for example: recharging the battery of the power bank 1 with a connecting cord that connects with a USB connector. Also, the recharging current can be either 3000 mA or 8000 mA to achieve the fast recharging effect. In addition, the power bank 1 includes the switch 7 and the function indicator light 8 that are disposed on the front panel 1c of the power bank 1, so that the power bank 1 can be turned on/off and selected different function modes, in which the function mode can be a low power consumption selection mode, and the switch 7 of the power bank 1 and the function indicator light 8 can be connected to the control module 12 (shown in FIG. 3). Furthermore, a touch switch 9 can be selectively configured on the side adjacent to the front panel 1c of the power bank 1 (the illustration of the touch switch 9 refers to FIG. 2).

Furthermore, please refer to FIG. 2, which is a schematic view of a specific embodiment of a beauty device for the present invention. As shown in FIG. 2, the beauty device of the present invention is composed by a power bank 1 and at least one beauty instrument 3, 4, 5, 6, in which, the power bank 1 have the ability to provide various power supply specifications to the beauty instruments 3, 4, 5, 6. The power bank 1 can provide various power supply specifications to the beauty instruments 3, 4, 5, 6 through the connecting interface 10 (as shown in FIG. 1). In addition, each of the beauty instruments 3, 4, 5, 6 includes a head 31, 41, 51, 61 and the connecting portion 2 opposite to the head. Also, inside of the beauty instrument 3, 4, 5, 6 has a beauty function output module 24 (as shown in FIG. 3) which connects with the head 31, 41, 51, 61 and the connecting portion 2 respectively. The head 31, 41, 51, 61 of the beauty instrument 3, 4, 5, 6 has various types and functions, such as: the head 31 of the T-shaped massage head 3, the head 41 of the scalp massage head 4, the head 51 of the eye massage head 5, or the head 61 of the color light massage head 6. The connecting portion 2 of each beauty instrument 3, 4, 5, 6 can be a USB connector. In addition, FIG. 2 also shows that the power bank 1 includes a switch 7 and a recharging interfacell. Obviously, the power bank 1 can be turned on by the switch 7 through connecting with the connecting interface 10 and connection portions 2 of each of the beauty instruments 3, 4, 5, 6, such that each different beauty instrument 3, 4, 5, 6 can receive the corresponding power supply specification from the power bank 1.

Please continue to refer to FIG. 2. FIG. 2 shows that when the touch switch 9 is configured on the side adjacent to the front panel of the power bank 1. In this embodiment, each beauty instruments 3, 4, 5, 6 includes a head 31, 41, 51, 61 and the connecting portion 2 opposite to the head 31, 41, 51, 61. The beauty instrument 3, 4, 5, 6 has a beauty function output module 24 (shown in FIG. 3) and is respectively connected to the head 31, 41, 51, 61 and the connecting portion 2. The head 31, 41, 51, 61 of the beauty instrument 3, 4, 5, 6 has various types and functions, such as: the head 31 of the T-shaped massage head 3, the head 41 of the scalp massage head 4, the head 51 of the eye massage head 5, or the head 61 of the color light massage head 6. The connecting portion 2 of each beauty instrument 3, 4, 5, 6 is USB connector. Particularly, the entire head 31, 41, 51, 61 of each beauty instrument 3, 4, 5, 6 preferably is covered by a metal layer or a part of the head 31, 41, 51, 61 of each beauty instrument 3, 4, 5, 6 is covered by a metal layer. The metal layer can be electrically connected to the connecting portion 2 of the beauty instrument 3, 4, 5, 6. The parts covered by these metal layers should be ensured that the entire beauty instrument 3, 4, 5, 6 can be in contact with the human body when the user uses the beauty device. For example, the entire head 31 of the T-shaped massage head 3 is covered by the metal layer, with the head 41 of the scalp massage head 4,could just selectively cover the protrusion 43 of the head of the scalp massage head 4by the metal layer, the same with the head 51 of the eye massage head 5, the protrusion 53 of the head 51 of the eye massage head 5 could be selectively covered by the metal layer, and either the entire head or partial of the head 61 of the color light massage head 6 can be covered by a metal layer. The main purpose of the metal layers electrically connected the connecting portion 2 on the end of the beauty instrument 3, 4, 5, 6 is to achieve power saving function and its operation method will be described later.

Next, please refer to FIG. 3, which is a block diagram showing a specific embodiment of the beauty device of the present invention. As shown in FIG. 3, the function module in the power bank 1 includes a control module 12 and a power output module 14, in which the power output module 14 is connected the control module 12. The power output module 14 is composed by a power management module 16 and a battery module 18, in which the power management module 16 can provide various power supply specifications respectively. The control module 12 and the power management module 16 can be an integrated chip or a chip combination. In addition, the control module 12 can also be connected to the touch switch 9 on the power bank 1. The function modules in each beauty instrument 3, 4, 5, 6 includes an identification module 22 and a beauty function output module 24, and the beauty function output module 24 is connected to the identification module 22 and the identification module 22 can record the power supply specification of the beauty function output module 24. The beauty function output module 24 is connected to the power output module 14, and the power output module 14 can provide power supply required by the beauty instrument 3, 4, 5, 6 during operation. The identification module 22 can be a Bluetooth module so that the power supply specifications can be transmitted. The beauty function output module 24 can be a vibration motor or a vibration device. Therefore, the control module 12 can control the battery module 18 to provide different power supply specifications to the beauty instrument 3, 4, 5, 6 through the power management module 16 in the power output module 14 since the beauty function output module 24 of each beauty instrument may have different power supply specifications. Therefore, in preferred embodiments, each beauty instrument 3, 4, 5, 6 can configure different identification module 22 and the identification module 22 is connected to the beauty function output module 24. When the identification module 22 is a Bluetooth module, it can be matched with different power supply specifications of the power management module 16 through the Bluetooth module (identification module 22) prior to use, when the power bank 1 uses the USB connector as the connecting interface 10 and is connected to the connection portion 2 of each beauty instrument, the control module 12 can immediately control the power management module 16 to provide appropriate power supply specification to the beauty instrument 3, 4, 5, 6. According to the embodiment, the power bank 1 of the present invention can provide different power supply specifications to the beauty function output modules of various beauty instruments 3, 4, 5, 6 through the control module 12 and the power management module 16, thereby, various beauty instruments 3, 4, 5, 6 can be activated, such that the single-function power bank 1 becomes a multi-functional power bank 1, which greatly meet the needs of user for the product use.

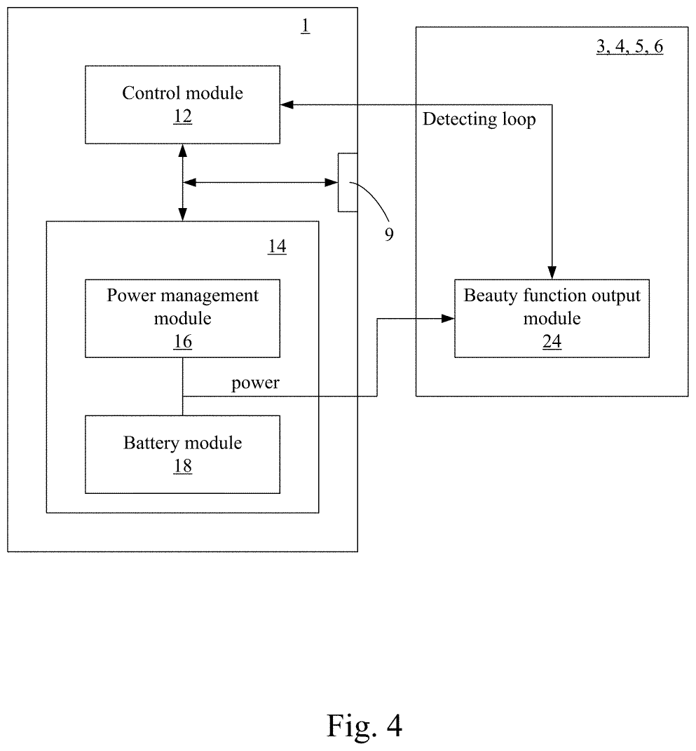

Next, please refer to FIG. 4, which is a block diagram showing another embodiment of the beauty device of the present invention. The function modules of the power bank 1 in FIG. 4 is the same as FIG. 3, as described in prior embodiment, therefore will not be described again. The difference is that the identification module 22 is not configured in the function module in beauty instrument in FIG. 4. In this embodiment, when the power bank 1 is connected to the connecting portion 2 of each beauty instrument 3, 4, 5, 6 through the connecting interface (USB connector) 10, the control module 12 will detect the circuits of the beauty function output module 24 in the beauty instrument 3, 4, 5, 6 to confirm the power supply specifications required to activate the beauty function output module 24 and after confirming the power supply specifications, the control module 12 can control the power management module 16 to provide the power supply according to the specifications required by various beauty instrument 3, 4, 5, 6

Similarly, according to the present embodiment, the power bank 1 of the present invention can provide power supply to various beauty function output modules with different power specification through the control module 12 and the power management module 16 in order to activate different beauty instruments 3, 4, 5, 6 so that the single-function power bank 1 can become multi-functions power bank 1, with these multiple combinations of functions, the needs of the users can be greatly met. In the embodiment of FIG. 3 and FIG. 4 mentioned above, when the control module 12 detects the power supply specification required by the beauty instrument 3, 4, 5, 6, for example, the control module 12 detects that the power supply specification of the T-type massage head 3 is the first type of the power supply specification, detects that the power supply specification of the eye massage head 5 is the second type of the power supply specification and detects that the scalp massage head 4 is the third type of the power supply specification, the control module 12 can control the power management module 16 to provide power supply according to each power supply specifications of the T-type massage head 3, the scalp massage head 4 and the eye massage head 5.

Finally, please return to FIG. 2 again. In the embodiment of the present invention, a design concept that is capable of achieving energy saving is provided. In an embodiment of the present invention, a design which can temporarily suspend the power supply is provided when the beauty device is in use. On one hand, since the entire head or part of the head of each beauty instrument 3, 4, 5, 6 is covered by a metal layer, the metal layer can be electrically connected to the connecting portion 2 of the beauty instrument 3, 4, 5, 6.

On the other hand, since the control module 12 is connected to the touch switch 9 which is configured on the front panel 1c of the power bank 1. Obviously, when the power bank 1 is connected to the connecting portion 2 of each beauty instrument 3, 4, 5, 6 through the connecting interface (USB connector) 10, the overall circuit system will construct two open circuits on the metal layer of the head of the beauty instrument 3, 4, 5, 6 and the touch switch 9 of the power bank 1. At this time, even if the power switch 7 has been switched to “ON”, the metal layer of the head 31, 41, 51, 61 of the beauty instrument 3, 4, 5, 6 and the touch switch 9 of the power bank 1 are still in an open circuit state, therefore, the power bank 1 at this time does not provide the power supply to the beauty function output module 24. When the user holds the power bank 1 by hand, the skin of the palm has been in contact with the touch switch 9 of the power bank 1. At this time, the power bank 1 still does not provide power to the beauty function output module 24, until the metal layer of the head of the beauty instrument 3, 4, 5, 6 is in contact with the human body or human face, when both open circuits are in contact with the human body and the power bank 1 form a loop by the resistance provided by the human body. At this time, the power bank 1 provides a power supply specification to the beauty function output module 24.

Therefore, when the users use the beauty instrument 3, 4, 5, 6 to perform the treatment and when the metal layer of the head of the beauty instrument 3, 4, 5, 6 is away from the human body' or human face, an open circuit is formed at the end of the metal layer of the head 31, 41, 51, 61 of the beauty instrument 3, 4, 5, 6, at the same time, the power bank 1 will stop providing the power supply. With this design, the goal of energy saving can be achieved.

According to the descriptions mentioned above, the multi-function power bank 1 of the present invention can provide the negative ion technology to promote absorption, the micro-shock technology to have soothing massage and the color light technology to whiten the skin. Also, through connecting with the connecting interface (USB connector) 10 of the power bank 1, it can be connected to fans, LED mirror for assistant light exposures, nail filing instrument for nail polishing, flashlights, vibration shaving razors, sound and light toys, negative ion fans, negative ion generators, LED mirror with magnifying function, Bluetooth speakers, etc., but not limited to the aforementioned functions. With the combinations of technology of Negative ion, micro-shock technology, color light massage and the power bank 1 mentioned above, it can solve the problems of a single function beauty instrument. The main problems solved are:
1: Use negative ion technology to promote the absorption of collagen and elastin in plant essence to achieve the purpose of removing wrinkles.
2: Use micro-shock massage technology to accelerate the microcirculation in the skin and to bring more nutrients to cell and capillary through the lymph, also to remove the accumulated toxins.
3: Use red light technology with a wavelength of 630 nm to whiten the skin effectively.
4: The power bank 1 can provide power supply to the beauty instrument 3, 4, 5, 6 at any time.

The invention applies the negative ion technology, the micro-shock technology or the color light massage head and power bank 1 to supply power for the facial cares, the dark circles under the eyes and the scalp area cares and also transform from single function to multiple combinations of functions, the negative ion technology helps to promote the absorption of the skin care products, the micro-shock technology assists to massage the skin, color light technology helps to whiten the skin, alleviate dark circles under the eyes and wrinkles effectively and improve hair quality as well. Also, it has the function to supply power from power bank 1, to provide power at any time for various electronic devices, with multiple combinations of functions, the needs of users can be met greatly.

The above description is the preferred embodiment of the patent, and it is not intended to limit the scope of the patent. Any equivalent structures or equivalent processes transformation made by using the contents of the specifications and the drawings, which is directly or indirectly applied to the related technical fields, is within the scope of this patent.

1. A beauty device, comprising:
a power bank, comprising:
a connecting interface, configured on one side of the power bank;
a touch switch, which is configured to the side adjacent to the connecting interface; and
a control module, which is configured inside of the power bank, and is connected to the connecting interface and the touch switch;
a power output module, which is configured inside the power bank and is connected to the control module, the power output module is composed of a power management module and a battery module, and the power management module is connected to the battery module,
wherein the power management module is configured to offers multiple power supply specifications of different powers; and
a beauty instrument, comprising a head and a connecting portion opposite to the head, the beauty instrument has a beauty function output module and is connected with both the head and the connecting portion, a metal layer is further configured on the head, and the metal layer is connected to the connecting portion,
thereby, the connecting portion of the beauty instrument is connected to the connecting interface of the power bank, and the power management module in the power output module supplies an appropriate power according to the power supply specification of the beauty instrument.

2. The beauty device according to claim 1, wherein the connecting interface of the power bank and the connecting portion of the beauty instrument are a USB interface.

3. The beauty device according to claim 1, wherein the power bank further comprise a charging interface, which is configured on the other end of the connecting interface of the power bank, and the charging interface is connected to the control module.

4. The beauty device according to claim 1, wherein the beauty instrument further comprise an identification module, the identification module is connected to the beauty function output module.

5. The beauty device according to claim 1, wherein the identification module is a Bluetooth device.

6. A beauty device, comprising:
a power bank, comprising:
a connecting interface, configured on one side of the power bank;
a control module, which is configured inside the power bank, and is connected to the connecting interface; and
a power output module, which is configured inside the power bank and is connected to the control module, the power output module is composed of a power management module and a battery module, and the power management module is connected to the battery module; and
a beauty instrument, comprising a head and a connecting portion opposite to the head, the beauty instrument has a beauty function output module and is connected with both the head and the connecting portion;
thereby, the connection portion of the beauty instrument is connected to the connecting interface of the power bank, initiating the power management module in the power output module to provide an appropriate power specification to the beauty function output module of the beauty instrument.

7. The beauty device according to claim 6, wherein the connecting interface of the power bank and the connecting portion of the beauty instrument are a USB interface.

8. The beauty device according to claim 6, wherein the power bank further comprise a charging interface, which is configured on the other end of the connecting interface of the power bank, and the charging interface is connected to the control module.

9. The beauty device according to claim 6, wherein the beauty instrument further comprise an identification module, the identification module is connected to the beauty function output module.

10. The beauty device according to claim 9, wherein the identification module is a Bluetooth device.

11. A beauty device, comprising:
a power bank, possess a top side and a bottom side, comprising:
a connecting interface, configured on the top side of the power bank;
a charging interface, configured on the bottom side of the power bank;
a touch switch, which is configured to the side adjacent to the connecting interface;
a control module, which is configured inside of the power bank, and is connected to the connecting interface and the touch switch respectively;
a power output module, which is configured inside the power bank and is connected to the control module, the power output module is composed of a power management module and a battery module, and the power management module is connected to the battery module,
wherein the power management module is configured to provide a plurality of different power specifications; and
a plurality of beauty instruments, each of which comprise:
a head, configured by a massage head;
a connecting portion, relative to the head set on the beauty instrument;
a beauty function output module, the beauty function output module is connected to the power output module, power is provided by the control module and the power management module for the beauty function output module; and
an identification module, used to record a power specification for the beauty function output module and is configured to pair with the power management module;
wherein, when the connecting interface of the power bank is connected to the connecting portion of the beauty instrument, by detecting a circuit on the control module confirms a power specification for the beauty function output module, wherein enable the control module to control the power specification corresponding to the beauty function output module to drive each of the beauty instruments.

12. The beauty device according to claim 11, wherein the connecting interface of the power bank and the connecting portion of the beauty instrument are a USB interface.

13. The beauty device according to claim 11, wherein the head can be a T-shaped massage head, a scalp massage head, an eye massage head or a color light massage head.

14. The beauty device according to claim 11, wherein the head or partial of the head of the beauty instrument is covered by a metal layer.

15. The beauty device according to claim 14, wherein the metal layer is connected to the connecting portion.

16. The beauty device according to claim 11, wherein the power specification of each of the plurality of beauty instruments may be the same or different.

17. The beauty device according to claim 11, wherein the identification module is a Bluetooth device.
